(19) United States
(12) Patent Application Publication
(10) Pub. No.: US 2011/0137334 A1
(43) Pub. Date: Jun. 9, 2011

(54) Electroactively Deployed Filter Device

(75) Inventors: James Anderson, Fridley, MN (US); Benjamin Arcand, Minneapolis, MN (US); Kyle Hendrikson, Litchfield, MN (US); Allen UtKe, Andover, MN (US)

(73) Assignee: Boston Scientific Scimed, Inc., Maple Grove, MN (US)

(21) Appl. No.: 12/631,513

(22) Filed: Dec. 4, 2009

(51) Int. Cl. A61M 29/00

(52) U.S. Cl. 606/200

(57) ABSTRACT

A medical device for capturing emboli from a blood vessel. An example medical device may include an elongated guide member. The elongated guide member may include a proximal end, a distal end, a first conductive lead, and a second conductive lead. The medical device may also include a power source connected to the first conductive lead and the second conductive lead. The medical device may also have a filter having a proximal end and a distal end, wherein the proximal end is coupled to the first and second conductive leads. The activation of the power source may transition the filter from a first compressed shape to a second expanded shape.

### FIELD OF THE INVENTION

The present disclosure relates generally to the field of medical filters, and more specifically, to vascular filter devices that are configured for percutaneous insertion into a blood vessel of a patient and deployment using electrical energy.

### BACKGROUND

Human blood vessels often become occluded or blocked by plaque, thrombi, other deposits, or material that reduce the blood carrying capacity of the vessel. Should the blockage occur at a critical place in the circulatory system, serious and permanent injury, and even death, can occur. To prevent this, sonic form of medical intervention is usually performed when significant occlusion is detected.

Several procedures are now used to open these stenosed or occluded blood vessels in a patient caused by the deposit of plaque or other material on the walls of the blood vessels. Angioplasty, for example, is a widely known procedure wherein an inflatable balloon is introduced into the occluded region. The balloon is inflated, dilating the occlusion, and thereby increasing the intraluminal diameter.

Another procedure is atherectomy. During atherectomy, a catheter is inserted into a narrowed artery to remove the matter occluding or narrowing the artery, i.e., fatty material. The catheter includes a rotating blade or cutter disposed in the tip thereof. Also located at the tip are an aperture and a balloon disposed on the opposite side of the catheter tip from the aperture. As the tip is placed in close proximity to the fatty material, the balloon is inflated to force the aperture into contact with the fatty material. When the blade is rotated, portions of the fatty material are shaved off and retained within the interior lumen of the catheter. This process is repeated until a sufficient amount of fatty material is removed and substantially normal blood flow is resumed.

In another procedure, stenosis within arteries and other blood vessels is treated by permanently or temporarily introducing a stent into the stenosed region to open the lumen of the vessel. The stent typically comprises a substantially cylindrical tube or mesh sleeve made from such materials as stainless steel or nitinol. The design of the material permits the diameter of the stent to be radially expanded, while still providing sufficient rigidity such that the stent maintains its shape once it has been enlarged to a desired size.

Unfortunately, such percutaneous interventional procedures, i.e., angioplasty, atherectomy, and stenting, often dislodge material from the vessel walls. This dislodged material can enter the bloodstream, and may be large enough to occlude smaller downstream vessels, potentially blocking blood flow to tissue. The resulting ischemia poses a serious threat to the health or life of a patient if the blockage occurs in critical tissue, such as the heart, lungs, kidneys, or brain, resulting in a stroke or infarction.

In general, existing devices and technology have a number of disadvantages including high profile, difficulty using multiple parts and components that result in an involved procedure, manufacturing complexity, and complex operation of the device or system.

### BRIEF SUMMARY

Embodiments of the present disclosure provide systems, methods, and devices for overcoming the above-referenced problems. More specifically, embodiments of the present disclosure include filter devices that have small, low, or no profiles, few parts and components, and are simple to manufacture and use. Consequently, embodiments of the present disclosure are able to be easily inserted into a patient, be steerable through the tortuous anatomy of a patient, provide filtering capabilities, have a sufficiently low profile to provide exchange capability so other medical devices can be advanced along the filter device, and be capable of removing the captured material without allowing such material to escape during filter retrieval.

According to one aspect of one embodiment of present disclosure, an illustrative embodiment of the present disclosure includes a medical device for capturing emboli from a blood vessel. This device includes an elongated guide member, such as a guidewire or hypo-tube having a lumen that extends from a distal end toward a proximal end thereof. The elongated guide member also includes a first conductive lead and a second conductive lead connected to a power source.

The medical device includes a filter having a proximal end and a distal end, where the proximal end is coupled to the first and second conductive leads. The filter transitions from a first compressed shape to a second expanded shape when the power source is activated.

In another configuration, an elongated guide member including a proximal end, a distal end, a first conductive lead and a second conductive lead; where a power source is connected to the first conductive lead and the second conductive lead.

The medical device includes an electrically activated actuating wire connected to the first and the second conductive leads; and an expandable filter assembly disposed distally from the elongated guide member. This device also includes a releasable restrainer holding the expandable filter in a first compressed position, where the releasable restrainer is coupled to the electrically activated actuating wire. Triggering the power source decouples the electrically activated actuating wire from the releasable restrainer, shifting the filter assembly from the first compressed position to a second expanded position.

In yet another configuration, a guide member having a proximal end, a distal end, a first conductive lead, and a second conductive lead; is connected to a power source.

The device includes an electrically activated restrainer disposed at the distal end of the guide member, where the electrically activated restrainer is connected to the first conductive lead and the second conductive lead. The electrically activated restrainer shifts from a first contracted configuration to a second expanded configuration when the power source is activated.

The medical device also includes an expandable filter disposed about the electrically activated restrainer, where the expandable filter shifts from a first compressed position to a second expanded position when the electrically activated restrainer shifts to the second expanded configuration.

These and other objects and features of the present invention will become more fully apparent from the following description and appended claims, or may be learned by the practice of the invention as set forth hereinafter.

### BRIEF DESCRIPTION OF THE DRAWINGS

FIG. 1a is a side view of an example medical device;

FIG. 1b is a cross-section of an example guide member;

FIG. 2 is a side view of another example medical device;

FIG. 3 is a side view of another example medical device;

FIG. 4 is a side view of another example medical device;

FIG. 5 is a side view of another example medical device;

FIG. 6 is a side view of another example medical device;

FIG. 7 is a side view of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE PREFERRED EMBODIMENTS

The present disclosure generally relates to percutaneous filter devices, systems, and methods of using the same. Embodiments of the present invention can be utilized in association with devices, systems, and methods for inserting a filter device, such as but not limited to a vascular filter device, within any blood vessel of a patient.

One or more of the embodiments of the filter devices of the present invention meet criteria for both guidewires and filter devices. For instance, it is preferable that a guidewire is steerable. Consequently, embodiments of the filter device of the present invention can be insertable within any blood vessel of a patient, such as but not limited to, coronary artery, carotid arteries, renal arteries, bypass grafts, superficial femoral artery, the arteries of the upper and lower extremities, or cerebral vasculature, and manipulated and steered by a physician to traverse the tortuous anatomy of the patient to a lesion or occlusion.

To assist the physician with the above-recited endeavor, one or more embodiments of the filter device include a shapeable, soft, distal tip. In addition, the filter device is capable of translating rotational movement or force applied to the proximal end thereof substantially equally to the distal end. In other words, with the filter device positioned within a vessel of the patient, as a physician rotates the proximal end of the filter device, the distal end of the filter device rotates substantially simultaneously with the movement of the proximal end. This is typically defined as having a one-to-one torqueability.

Further, the filter device of the present invention is kink resistant and is capable of receiving a variety of different coatings to provide electrical insulation, improve lubricity, have anti-thrombogenic properties, and/or reduce platelet aggregation.

With respect to the filter of the filter device of the present invention, in one embodiment, the filter is configured to capture material of a variety of sizes and enable removal of the captured material. Therefore, filter pore sizes and shapes can be selected based upon the size of material to be captured. The material can include but is not limited to particulates, thrombi, any atherosclerosis or plaque material dislodged during a procedure, or other foreign material that may be introduced in to the vasculature of the patient.

As discussed in greater detail below, filter frame 34 is adapted to have a reduced profile in a first compressed configuration, and a second expanded configuration. Such features are desirable when advancing medical devices through tortuous anatomy. Additionally, reducing (or completely removing) the number of mechanical parts within the guide member can further reduce the profile thereof.

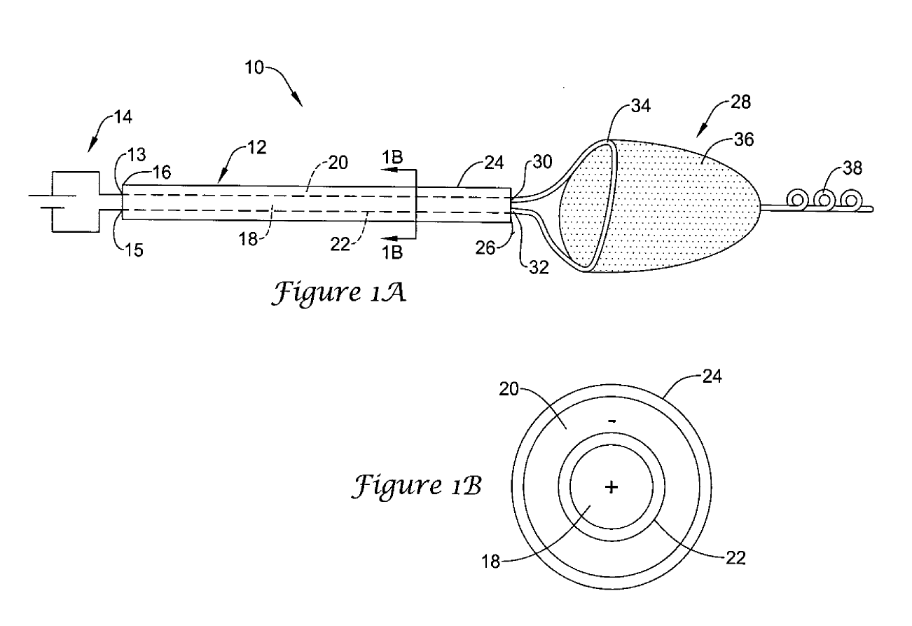

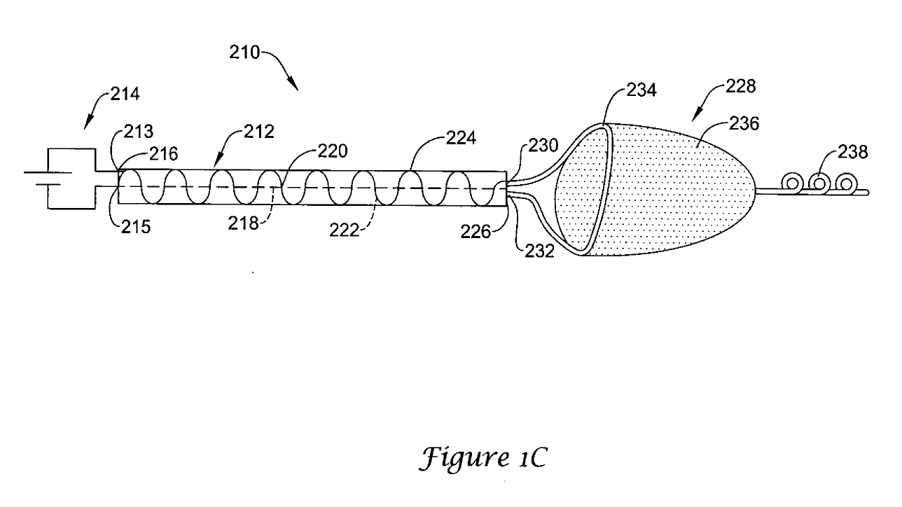

Referring now to FIG. 1A, depicted is one embodiment of a vascular filter device, designated by reference number 10, of the present disclosure. As illustrated, filter device 10 includes a guide member 12 having a distal end 26 and a proximal end 16. Extending between distal end 26 and proximal end 16 of guide member 12 are a first conductive lead 18 and a second conductive lead 20. The first 18 and second 20 conductive leads can consist of conductive wires running longitudinally or helically along guide member 12. For example, FIG. 1C illustrates an alternate embodiment wherein at least one of the conductive leads 220 forms a helix surrounding guide member 212, the helical conductive lead 220 can also be coated with an insulating material 224. The first conductive lead 18 and the second conductive lead 20 can also be embedded to guide member 12. Alternatively, guide member 12 can be used as an electrical conductor. For example, the components of guide member 12 can be manufactured by extrusion processes, followed by fully or in part coating their surface with an electrical insulator. FIG. 1B illustrates a multilayered guide member 12 wherein the first conductive lead 18 is insulated from the second conductive lead 20 by insulating layer 22. Furthermore, the second conductive lead 20 can also be coated with insulating coat 24 to enhance electrical conductance, improve lubricity, add anti-thrombogenic properties, reduce platelet aggregation, and protect electro-sensitive tissues.

The insulating coat may be made from a polymer or any other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the guide member 12 (including, for example, the surface of the first 22 and second 24 conductive leads) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of guide member 12, or other portions of device 10. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

As illustrated in FIG. 1A, a power source 14 is connected to the first 18 and second 20 connective leads. This connection can be achieved by including a first power connector 13 and a second power connector 15 between the power source 14 and the first 18 and second 20 connective leads. The connector to the first connective lead 13 and the connector to the second connective lead 15 can be located proximally, distally, or alongside guide member 12. Consequently, power source 14 can also be positioned proximally, distally or anywhere along guide member 12. Power source 14 can be any device or method capable of generating an electrical current; these may include, but are not limited to, batteries, charged capacitors, or any other source of electromagnetic energy.

Filter device 10 can be used to filter particulates, thereby acting or providing embolic protection during a procedure. Distal end 26 of guide member 12 includes a filter 28 including a frame 34, and a filter membrane 36. Filter 28 includes a first filter connector 30 and a second filter connector 32 configured to connect to the first connective lead 18 and the second connective lead 20 respectively. Filter frame 34 can be fabricated from memory material which can confer martensitic and autenistic properties to the filter. For example, the embodiment illustrated in FIG. 1A shows filter 34 in its autensite expanded shape. Filter frame 26 can be fabricated such that the autenistic finish temperature is above the intracorporal temperature.

Disposed upon the filter 28, is a coil tip 38 that is commonly used with guidewires, hypo-tubes, and other medical devices. This coil tip 38 may be configured to allow a physician or clinician to shape the same before insertion into a body lumen. In this manner, the physician or clinician is able to configure the tip with an appropriately shaped J that enables guide member to be guided through the tortuous anatomy of a patient. The coil tip 38 can be manufactured from platinum, platinum alloys, radiopaque materials, metals, alloys, plastic, polymer, synthetic material, combinations thereof, or other materials that provide an appropriate radiopaque signature, while capable of being shaped by a physician or clinician.

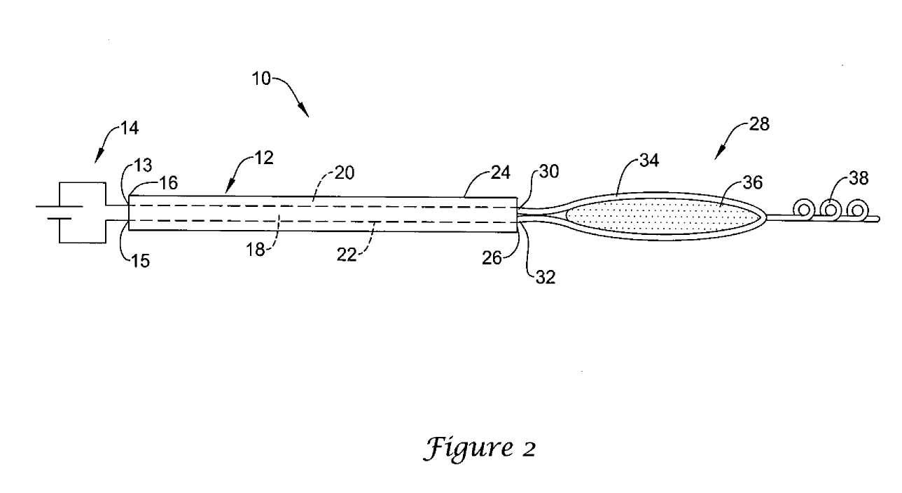

FIG. 2 illustrates a side view of filter 28 on its compressed martensite shape. A transition from the martensite state to the austenite state can be obtained by triggering the power source 14 and passing a current through filter frame 34 capable of generating enough heat (given the intrinsic electrical resistance of the material used to manufacture the frame) to reach the austentite finish temperature. For example, the austentite finish temperature of filter 28 can be any temperature above the normal body temperature.

In this configuration, filter device 10 is capable of being insertable into any blood vessel of a patient or body and function as a guidewire or exchange wire for other medical components or devices, such as but not limited to catheters, stents, balloons, atherectomy devices, or other components or devices that can be exchanged using a guidewire.

Illustratively, the term “guide member” can refer to a member that is completely solid, such as a guidewire, a member that partially includes a lumen therein, or a member that includes a lumen extending from a proximal end to a distal end thereof, such as a hypo-tube. Consequently, the term “guide member” can include or encompass a guidewire or a hypo-tube that is configured to perform the functions described herein.

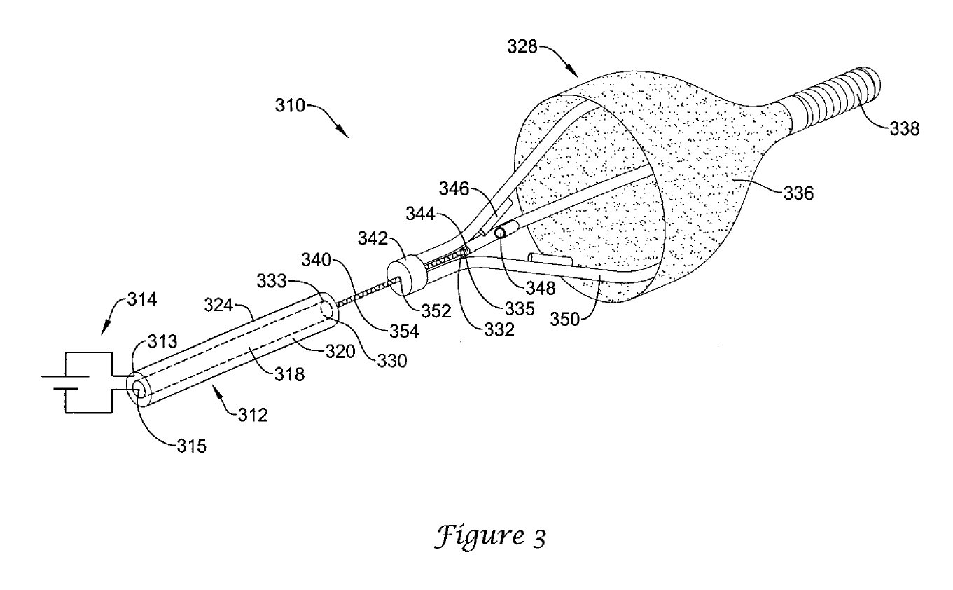

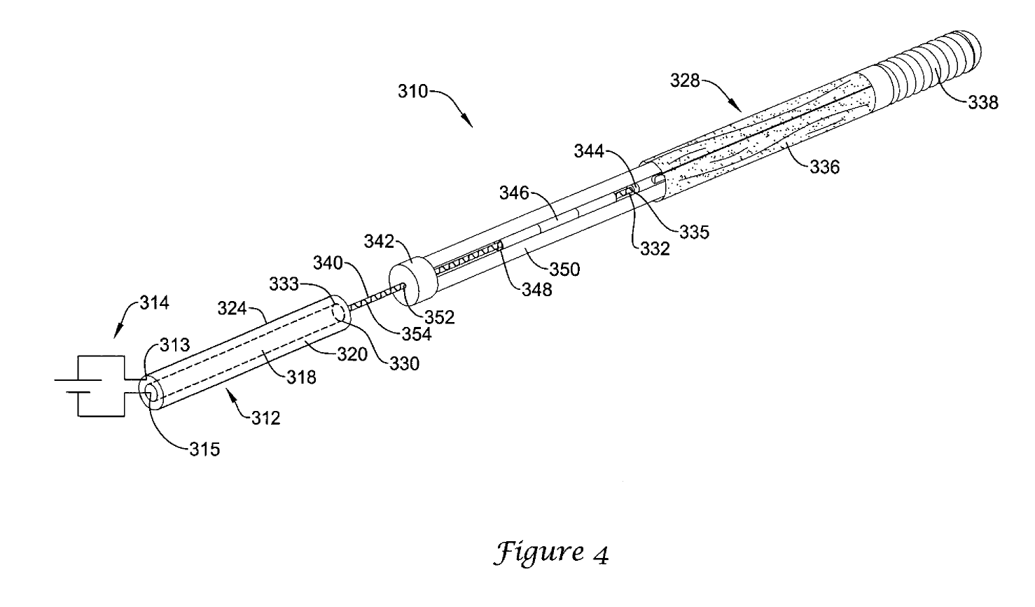

Referring now to FIG. 3, depicted is a side view of a filter device 310 comprising at least a strut 350 where each strut includes a loop, cylinder or comparable structure attached to said strut 350. The cylinders 346 are designed such that when a distally disposed filter 328 is collapsed, the lumen 348 of the cylinders 346 axially align forming a continuous longitudinal lumen 348 as shown in FIG. 4. The filter device also includes a hub 342 attached to the proximal end of the filter. The hub 342 includes a lumen 352 configured to receive an electrically activated actuating wire 340. Additionally, the struts 350 may be attached to said hub 342, providing structural soundness to the filter 328. The electroactivate wire 340 can be fully or in part fabricated from a variety of electroactive materials. For example, the electroactivate actuating wire 340 can be constructed from memory alloys like Nitinol, CuZnAl, and CuAlNi. Also, the electroactive actuating wire 340 can be manufactured from electroactive polymers such as ionic polymer gels, ionomeric polymer-metal composites, conductive polymers, and carbon nanotubes. Electroactive polymers are polymers whose shape is modified when a voltage is applied to them. They can be used as actuators or sensors. As actuators, they are characterized by being able to undergo a large amount of deformation while sustaining large forces.

Filter 328 can be located about the distal or proximal ends of guide member 312. Furthermore, filter 328 can taper distally or proximally depending on the location of filter 312. Additionally, guide member 312 can have multiple filters 328. The filters 328 can taper in the same orientation or opposite orientations. For example, multiple filters tapering in opposite orientations may allow embolic protection when atherosclerosis or plaque material is located nearby the elbow of a branching blood vessel.

Similar to the embodiments described above, the filter device 310 illustrated in FIG. 3 also comprises a power source 314 coupled to first 318 and second 320 conductive leads disposed about a guide member 312, wherein the conductive leads 318/320 can be connected alongside the guide member by leads connectors 313 and 315. Also, filter 328 disposed about the distal end 326 of guide member 312 may include a filtering membrane 336 and a coil tip 338.

As depicted in FIG. 4, the electrically activated actuating wire 340 is configured to engage the continuous lumen 348 formed by the aligned cylinders 346 when the filter 328 is compressed. Furthermore, the electrically activated actuation wire 340 can have a distal stop 344 with a diameter larger than the diameter of the most proximal cylinder lumen 348 preventing; the release of the actuation wire 340 before, during, and after actuation.

The electrically activated actuation wire 340 illustrated in FIG. 3 respectively connects to the first 318 and second 320 conductive leads at a first connection point 330 and a second connection point 332. To allow current to flow across the electrically activated actuation wire 340 a lead may connect to the proximal end 333 of the wire 340 and the other lead to the distal end 335 of the actuation wire 340. The most distal actuation wire conductive lead 354 can be embedded in the actuation wire 340 or it could helically surround the actuation wire.

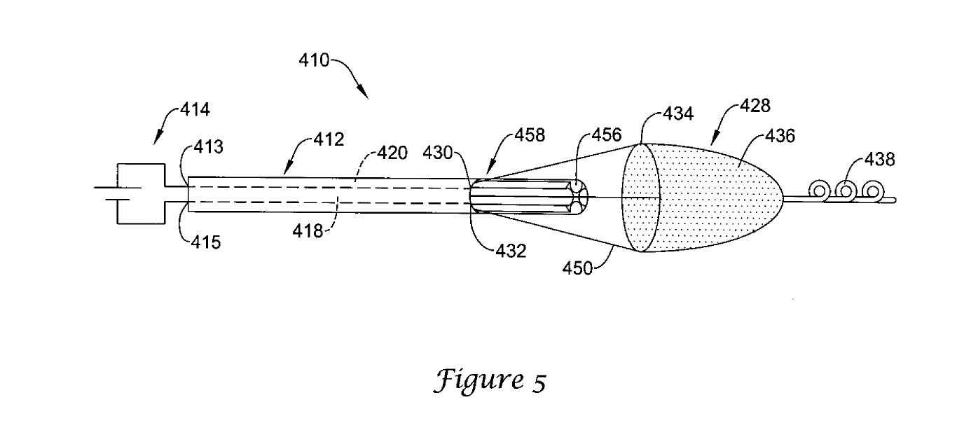

Moving now to FIG. 5, a side view of a medical device is illustrated. The medical device 410 includes a power source 414 and conductive leads 418/420 attached thereto by lead connectors 413/415. Said conductive leads can travel along the guide member 412 and connect to distally disposed electroactive restraining mechanism 458. This restraining mechanism 458 can be electrically coupled to the conductive leads 418/420 by conductive connection points 430/432. The restraining mechanism 458 can be constructed from electroactive polymers. Examples of electroactive polymers include, but are not limited to, ionic polymer gels, ionomeric polymer-metal composites, conductive polymers, and carbon nanotubes. The electrically activated restraining mechanism 458 may comprise a plurality of channels 456 running alongside the restraining mechanism 458. The medical device 410 may further include an expandable filter 434 disposed about the restraining mechanism 458. The expandable filter 434 can include a plurality of filter legs 450, a filter membrane 436 and a coil tip 438. The filter legs 450 can be disposed within the channels 456 of the restraining mechanism 458 when the restraining mechanism 458 is contracted. This restrains the legs of the filter 450 within the channels 456 of the restraining mechanism 458, maintaining the filter 434 in a compressed configuration.

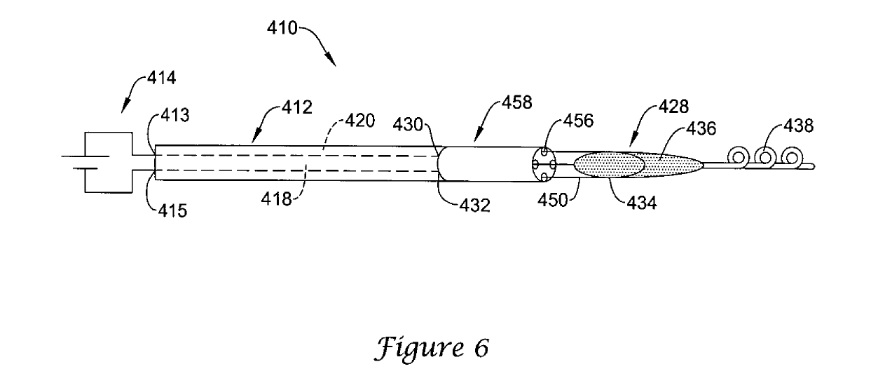

The electroactive retraining mechanism 458 can be configured to remain contracted when the power source 414 is inactive as depicted in FIG. 6. Alternatively, the electroactive restrain mechanism 458 may remain contracted as long as an electrical current is constantly offered by the power source 414. Consequently, if the operator deactivates or interrupts the current flow, the electroactive restraining mechanism 458 expands releasing the filter legs 450, and allowing the expandable filter 434 to transition from the compressed configuration to an expanded configuration as shown in FIG. 5.

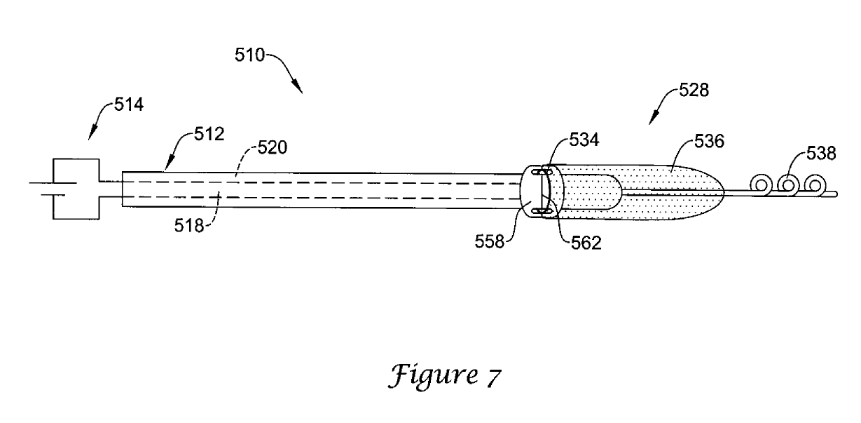

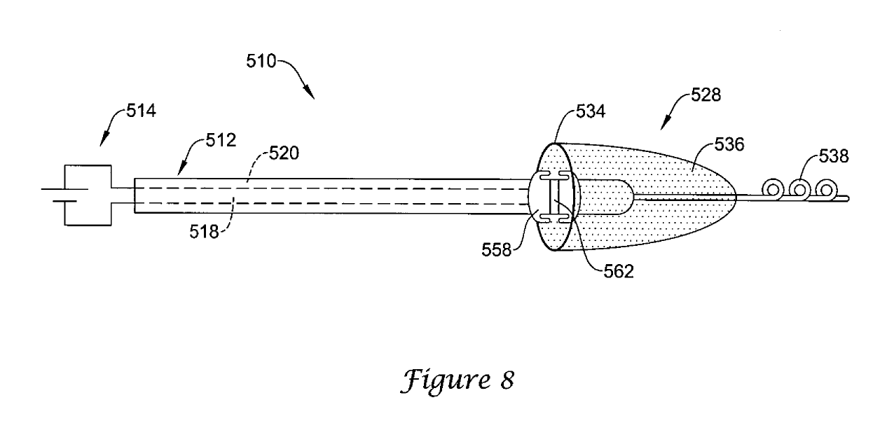

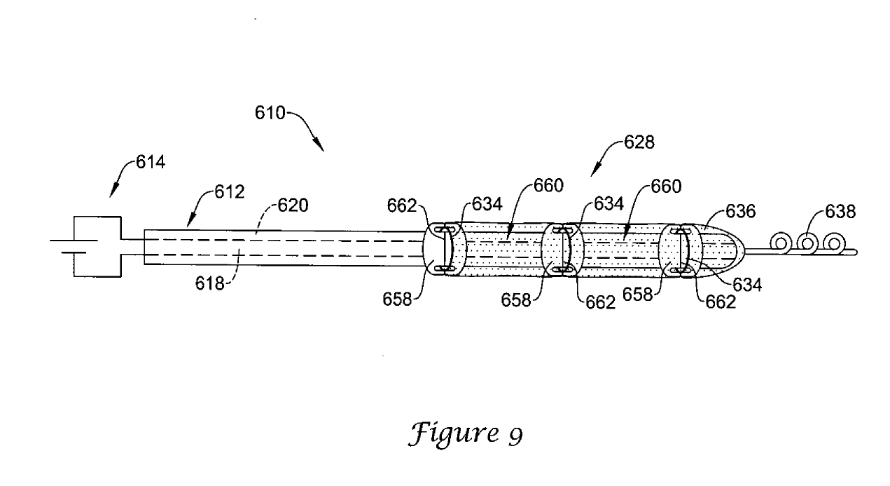

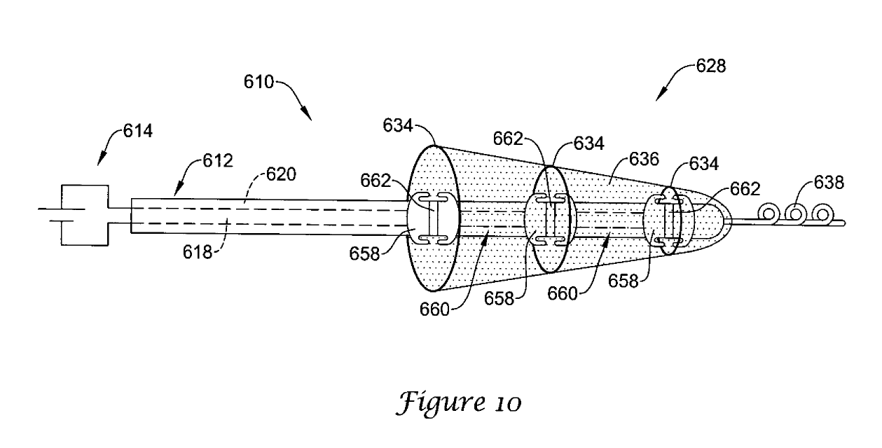

FIG. 7 depicts an electroactive restraining mechanism 558 having at least a perpendicularly oriented channel 562, allowing the restraining of embolic filters with a loop frame 534. Alternatively, the electroactive restraining mechanism 558 can be shaped like fingers or any similar structure capable of restraining a loop based filter frame 534. As in previous embodiments, the electroactive restraining mechanism 558 can be coupled to a power source 514 by conductive leads 518 and 520. The electroactive restraining mechanism 558 can be disposed distally of guide member 512, or along any location of the guide member 512. The filter 528 may further comprise a filter membrane 536 and a coil tip 538 attached thereto. This particular embodiment illustrates filter 528 in its compressed configuration, alternatively FIG. 8 illustrates a side view of the same device when filter 528 is expanded.

FIG. 9 illustrates a side view of a medical device comprising a plurality of electroactive restraining mechanisms 658 including perpendicularly oriented channels 662. The plurality of electroactive restraining mechanisms 658 can restrain strut of loop based frames. Activating power source 614 passes a current along conductive leads 618/620; this allows the deployment of one or multiple struts or loops 634 along the guide member 612 as illustrated in FIG. 10. The different electroactive restraining mechanisms 658 can be separated by guide member portions 660. These guide member portions 660 can be contracted with the same material as guide member 612. Alternatively, material with different elastic, steerability, and electromagnetical properties can be selected to confer desired properties. Also, the different electroactive restraining mechanisms 658 can have different elastic, steerability, and electromagnetical properties. For example, different activation thresholds for the electroactive restraining mechanisms 658 may allow selective activation of electroactive restraining mechanisms 658 and deployment of specific struts or loops 634 along guide member 612. Also, given the intrinsic conductance and/or electromagnetic properties of the electroactive restraining mechanisms 658, the electromagnetic properties of guide member portions 660 may be altered to obtain a desirable result. Conductive leads 618 and 620 can run along guide member 612, electroactive restraining mechanisms 658, and guide member portions 660. Depending on the level of resistance provided by the electroactive restraining mechanisms 658, insulating layers can be included therein to allow the conduction of current through the electroactive restraining mechanisms 658 to subsequent guide member portions 660. The use of multiple struts or hoops provides additional support for the deployment of larger filter membranes 636 attached thereto.

A potential problem of using large filter membrane is that it may have the propensity to collapse and/or become entangled. Using the multiple struts and/or hoops 634 allows the full and secure deployment of a filter membrane 636. Different portions of filter membrane 636 can have different shapes depending on the nature of the medical procedure. For example, the most proximal portions of filter membrane 636 can be shaped like a cylinder and the distal portions can taper distally. Alternatively, the different portions of filter membrane 636 can continuously taper distally. Moreover, filter membrane 636 can further comprise a coil tip 638. The combination of a conductive guide member 612 comprising conductive leads and electroactive materials allows multi-hoop/strut deployment while maintaining a narrow profile.

Independent deployment of struts/hoops is particularly difficult in purely mechanical based systems because of size limitations. For this reason, the use of conductive and electroactive materials confers numerous desirable features while maintain a narrow profile, allowing the use of these devices in deep artery intervention and other medical procedure wherein advancing such devices through a narrow lumen is required.

The guide members, and/or filters, and/or filter frames, and/or filter struts, and/or coil tips member, and the like may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the guide member, and/or filter, and/or filter frame, and/or filter struts, and/or coil tip member, and the like may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guide members in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the guidewire to achieve the same result.

In some embodiments, a degree of MRI compatibility is imparted into the guidewire. For example, to enhance compatibility with Magnetic Resonance Imaging (MRI) machines, it may be desirable to make guide members, and/or filters, and/or filter frames, and/or filter struts, and/or coil tips member in a manner that would impart a degree of MRI compatibility. For example, the guide member, or portions thereof, may be made of a material that does not substantially distort the image and create substantial artifacts (artifacts are gaps in the image). Certain ferromagnetic materials, for example, may not be suitable because they may create artifacts in an MRI image. The guide member, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A medical device for capturing emboli from a blood vessel, the medical device comprising:
an elongated guide member including a proximal end, a distal end, a first conductive lead, and a second conductive lead;
a power source connected to the first conductive lead and the second conductive lead;
a filter having a proximal end and a distal end, wherein the proximal end is coupled to the first and second conductive leads; and
wherein activating the power source transitions the filter from a first compressed shape to a second expanded shape.

2. The medical device of claim 1, wherein the conductive leads are embedded in the guide member.

3. The medical device of claim 1, wherein at least one the conductive leads consists of a wire alongside the guide member.

4. The medical device of claim 3, wherein the wire helically surrounds the guide member.

5. The medical device of claim 3, wherein the conductive leads of the guide member comprise layers.

6. The medical device of claim 1, wherein the power source is a battery.

7. The medical device of claim 1, wherein the power source is a charged capacitor.

8. The medical device of claim 1, wherein the filter is made from a memory material.

9. The medical device of claim 10, wherein the memory material is Nitinol.

10. A filter device for percutaneous insertion into a blood vessel, the filter device comprising:
an elongated guide member including a proximal end, a distal end, a first conductive lead and a second conductive lead;
a power source connected to the first conductive lead and the second conductive lead;
an electrically activated actuating wire connected to the first conductive lead and the second conductive lead;
an expandable filter assembly disposed distally from the elongated guide member;
a releasable restrainer holding the expandable filter in a first compressed position, wherein the releasable restrainer is coupled to the electrically activated actuating wire; and
wherein triggering the power source decouples the electrically activated actuating wire from the releasable restrainer, thereby shifting the filter assembly from the first compressed position to a second expanded position.

11. The filter device of claim 10, wherein, the expandable filter comprises a hub having a lumen and plurality of struts attached thereto, said struts each having an associated lumen, wherein the lumens of the plurality of struts may be aligned with the lumen of the hub.

12. The filter device of claim 11, wherein the electrically activated actuating wire is designed to engage the aligned lumens of the hub and the plurality of struts.

13. The filter device of claim 10, wherein the electrically activated actuating wire is made from a memory material.

14. The filter device of claim 10, wherein the electrically activated actuating wire is made from an electroactive polymer.

15. A cardiovascular medical device comprising:
a guide member having a proximal end, a distal end, a first conductive lead, and a second conductive lead;
a power source connected to the first and second conductive leads;
an electrically activated restrainer disposed at the distal end of the guide member, wherein the electrically activated restrainer is connected to the first conductive lead and the second conductive lead;
the electrically activated restrainer shifts from a first contracted configuration to a second expanded configuration when the power source is activated; and
an expandable filter having one or more filter legs disposed about the electrically activated restrainer, wherein the expandable filter shifts from a first compressed position to a second expanded position when the electrically activated restrainer shifts to the second expanded configuration.

16. The cardiovascular medical device of claim 15 wherein, the electrically activated restrainer is made from an electroactive polymer.

17. The cardiovascular medical device of claim 16 wherein, the electroactive polymer is an ionic polymer gel.

18. The cardiovascular medical device of claim 16 wherein, the electroactive polymer is an ionomeric polymer-metal composite.

19. The cardiovascular medical device of claim 15 wherein, the electrically activated restraining mechanism includes one or more channels.

20. The cardiovascular medical device of claim 19 wherein, the one or more filter legs are disposed in the one or more channels of the electrically activated restraining mechanism.
